(19) United States
(12) Patent Application Publication
(10) Pub. No.: US 2003/0171544 A1
(43) Pub. Date: Sep. 11, 2003

(54) Alcohol dehydrogenase and use thereof

(75) Inventors: Thomas Riermeier, Floersheim (DE); Uwe Bornscheuer, Greifswald (DE); Josef Altenbuchner, Nufringen (DE); Petra Hildebrandt, Weitenhagen (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/096,494

(22) Filed: Mar. 13, 2002

(30) Foreign Application Priority Data
Mar. 13, 2001 (DE) 101 12 401.5

(51) Int. Cl. C07H 21/02; C07H 21/04; C07K 1/00; C07K 14/00; C07K 17/00

(52) U.S. Cl. 530/350; 536/23.1

(57) ABSTRACT

The invention relates to a novel alcohol dehydrogenase (ADHF1) from Pseudomonas fluorescens (DSM 50106) and to functional variants thereof and to a process for selective reduction of ketones to the corresponding alcohols by using such alcohol dehydrogenases.

### DESCRIPTION

The invention relates to a novel alcohol dehydrogenase (ADH) from Pseudomonas fluorescens and also to a process for selective reduction of ketones to the corresponding alcohols by using such alcohol dehydrogenases.

The alcohol dehydrogenases (EC 1.1.1.1) belong to the group of oxidoreductases. Alcohol dehydrogenases catalyze a multiplicity of biological reactions in which alcohol substrates are oxidized to the corresponding ketones or aldehydes or in which the opposite reduction from aldehyde or ketone to alcohol is catalyzed. Alcohol dehydrogenase-mediated biological processes include such important reactions as the last step of alcoholic fermentation, i.e. conversion of glucose into ethanol in yeasts, the reduction of all-trans retinal to all-trans retinol (vitamin A₁) in the retina or the degradation of blood alcohol in the liver. The reactions described are normally reversible and take place in the presence of nicotinamide adenine dinucleotide (NAD⁺/NADH) or nicotinamide adenine dinucleotide phosphate (NADP⁺/NADPH) as coenzyme. In most alcohol dehydrogenases, zinc to which the substrate oxygen atom can coordinate serves as the catalytic center.

Apart from the crucial importance of alcohol dehydrogenases in biological processes, there are attempts to make use of these enzymes for organochemical synthesis to prepare alcohols, ketones or aldehydes. Of particular interest from a technical point of view is the enantioselective synthesis of optically active alcohols by catalytic reduction of the corresponding ketones. Up to now, especially alcohol dehydrogenases from horse liver, yeast (YADH) or from Thermoanaerobium brockil have been used in organic synthesis.

Since the individual alcohol dehydrogenases differ greatly with respect to their substrate specificity and their selectivity, there is a great interest in finding further alcohol dehydrogenases with novel enzymic properties. For this purpose, in recent years quite a number of alcohol dehydrogenases have been isolated from different organisms and characterized.

The vast majority of presently known alcohol dehydrogenases are from yeasts, and their substrate specificities vary greatly. Some of the ADHs identified to date are from Pseudomonas sp., such as, for example, a specific dehydrogenase described by Shen, G. J. et al. (Chem. Soc., Chem. Commun. 9, 677-679; 1990) from the strain ATCC 49688, which has low substrate tolerance, a Pseudomonas putida ADH converting allylic alcohols (Malone, V. F., Appl. Environm. Microbiol. 1999, 65, 2622-2630) or a relatively unspecific dehydrogenase with broad substrate acceptance, described by Bradshaw, C. W. et al. (J. Org. Chem. 57, 1526-1532; 1992). Up until now, however, it has been impossible to clone the known Pseudomonas sp. ADHs which are therefore not available for a broad application for organosynthetic purposes.

It is therefore the object of the present invention to provide novel alcohol dehydrogenases which are readily accessible and which can be used in organic synthesis for oxidizing alcohols to ketones or for reducing ketones to alcohols.

The present invention relates to a novel alcohol dehydrogenase (ADHF1) from Pseudomonas fluorescens (DSM 50106), having the following amino acid sequence (Seq. Id. No. 1)

(SEQ ID NO.1)

MKSFNGRVAA ITGAASGMGR ALALALAREG CHLALADKNA
QGLEQTLALI KTSTLSPVMV TTQVLDVADR QAMEAWAARC
VAEHGQVNLV FNNAGVALSS TVEGVDYADL EWIVGINFWG
VVHGTKAFLP HLKASGDGHV INTSSVFGLF AQPGMSGYNA
TKFAVRGFTE ALRQELDLQR CGVSATCVHP GGIRTDICRS
SRIDANMTGF LIHSEQQARA DFEKLFITDA DQAAKVILQG
VRKNKRRVLI GRDAYFLDLL ARCLPAAYQA LVVLASKRMA
PKQRRPVFET NDEPRL

or an allelic or functional variant thereof or a functional part sequence thereof.

A functional variant in accordance with the present invention means an alcohol dehydrogenase comprising an amino acid sequence having a sequence homology of more than 60%, preferably of more than 80%. In addition, a functional part sequence means alcohol dehydrogenases which contain preferably amino acid fragments of at least 50 amino acids, particularly preferably of more than 100 amino acids, but functional variants having deletions of up to 100 amino acids, preferably with up to 50 amino acids, are also included under the term “functional part sequence”.

In addition, the alcohol dehydrogenases of the invention may have posttranslational modifications such as, for example, glycosylations or phosphorylations.

The present invention further relates to nucleic acids coding for the alcohol dehydrogenases of the invention or to an allelic or functional variant thereof or to part sequences thereof or to DNA fragments which are complementary to those nucleic acid sequences hybridizing with coding nucleic acids under stringent conditions.

The alcohol dehydrogenase gene from Pseudomonas fluorescens (DSM 50106) contains the following coding nucleic acid sequence (Seq. Id. No. 2):

(SEQ ID NO:2)

ATGAAGTCAT TCAACGGCCG CGTGGCGGCG ATTACCGGCG
CGGCATCCGG CATGGGTCGC GCATTGGCCC TGGCACTCGC
GCGCGAAGGT TGCCACCTGG CACTGGCGGA CAAAAACGCC
CAAGGCCTGG AGCAGACCCT GGCACTGATC AAGACCTCGA
CCCTGTCGCC GGTGATGGTC ACCACCCAGG TGCTGGATGT
GGCCGACCGC CAGGCCATGG AGGCTTGGGC GGCGCGCTGC
GTGGCCGAGC ATGGCCAGGT CAACCTGGTG TTCAACAACG
CCGGCGTGGC CCTGTCGAGT ACGGTCGAAG GCGTGGACTA
CGCCGACCTG GAGTGGATCG TCGGCATCAA CTTCTGGGGC
GTGGTCCACG GCACCAAGGC GTTCCTGCCG CACCTCAAGG
CCAGCGGCGA CGGCCATGTG ATCAACACGT CCAGCGTGTT
CGGCCTGTTT GCCCAGCCCG GCATGAGCGG TTACAACGCG
ACCAAATTCG CCGTGCGCGG CTTTACCGAA GCCCTGCGCC
AGGAGCTGGA CCTGCAACGC TGCGGCGTCT CGGCCACCTG
CGTGCACCCC GGCGGCATCC GCACCGATAT CTGTCGCAGC
AGCCGCATCG ACGCGAACAT GACCGGCTTC CTGATCCACA
GCGAACAGCA GGCCCGCGCC GACTTCGAAA AACTCTTCAT
CACCGATGCC GACCAGGCCG CCAAGGTGAT CCTGCAAGGC
GTCCGCAAAA ACAAGCGTCG CGTGCTGATA GGCCGCGACG
CGTATTTCCT CGACCTGCTC GCCCGTTGCC TGCCGGCGGC
CTATCAAGCG CTGGTGGTGC TGGCCAGCAA GCGCATGGCC
CCCAAGCAAC GCAGGCCAGT GTTTGAAACC AACGACGAGC
CCCGTCTCTG A

Preferred nucleic acids of the invention include Seq. Id. No. 2 and allelic or functional variants thereof which are more than 50%, preferably more than 75%, particularly preferably more than 90%, homologous or part sequences thereof, preferably of at least 150 nucleotides, particularly preferably of at least 300 nucleotides, or DNA fragments which are complementary to those nucleic acid sequences hybridizing with a coding nucleic acid Seq. Id. No. 2 or an allelic or functional variant or part sequences thereof under stringent conditions. For this purpose, it is possible to utilize common hybridization conditions such as, for example, 60° C., 0.1×SSC, 0.1% SDS.

The information from Seq. Id. No. 2 may be utilized to generate primers in order to identify and clone directly allelic forms by means of PCR, for example in other Pseudomonas sp. strains. In addition, it is possible, due to the sequence information, to use probes for finding further naturally occurring functional variants of the adhF1 gene and thus the corresponding encoded enzyme variants. Starting from Seq. Id. No. 2 or from functional variants, allelic thereto or occurring naturally, it is possible, for example, to obtain via PCR a bank of artificially generated functional enzyme variants by using a faulty DNA polymerase.

The coding DNA sequences may be cloned into conventional vectors and, after transfecting host cells with such vectors, be expressed in cell culture. Examples of suitable expression vectors are pUC, pGEX or pJOE for E. coli, but it is also possible to use expression vectors of other prokaryotic unicellular organisms. Examples of expression vectors which have proved suitable for yeasts are the pREP vector and the pINT vector. Examples suitable for expression in insect cells are Baculovirus vectors as disclosed in EP-B1-0127839 or EP-B1-0549721 and for expression in mammalian cells SV40 vectors which are generally available. Particular preference is given to expression vectors for unicellular prokaryotic and eukaryotic organisms, in particular from the group consisting of pGEX, pJOE, pREP and pINT.

Apart from the usual markers such as, for example, ampicillin resistance, the vectors may contain further functional nucleotide sequences for regulating, in particular repressing or inducing, expression of the ADH gene and/or the reporter gene. Promoters which are preferably used are inducible promoters such as, for example, the rha promoter or the nmt1 promoter, or strong promoters such as, for example, the lac, ara, lambda, pL, T7 or T3 promoter. The coding DNA fragments must be transcribable in the vectors, starting at a promoter. Further examples of proven promoters are the Baculovirus polyhedrin promoter for expression in insect cells (see, for example, EP-B1-0127839) or the early SV40 promoter or the LTR promoters, for example of MMTV (Mouse Mammary Tumor Virus; Lee et al. (1981) Nature, 214, 228).

The expression vectors of the invention may contain further functional sequence regions such as, for example, an origin of replication, operators or termination signals.

The vectors described can be used for transforming host cells by conventional methods such as, for example, the PEG/DMSO method, or by electroporation.

As a result, the present invention further relates to expression systems comprising host cells or host cell cultures which are transfected with the vector systems just described. Preferred hosts are unicellular prokaryotic organisms, in particular E. coli. To express eukaryotic adh genes of the invention it may be advantageous to use eukaryotic expression systems in order to introduce, for example, posttranslational modifications typical for eukaryotes into the adh gene product. Particularly suitable eukaryotic host cells are yeasts.

A preferred expression system comprises an alcohol dehydrogenase gene according to Seq. Id. No. 2 or an allelic or functional variant or a part sequence thereof in a vector suitable for expression in E. coli such as, for example, a pJOE2775 vector, and the dehydrogenase gene must be cloned into said vector in such a way that it can be transcribed. In a particularly preferred embodiment, the introduced adh gene is fused in addition to a histidine tag provided by the vector. Preference is given to cloning the introduced adh gene into the pJOE2775 vector such that transcription is under the control of the rhamnose-inducible promoter present in the vector.

The expression systems may be cultured using standard protocols known to the skilled worker. Depending on transcription control and the vector used, expression of the gene introduced into the expression system may be either constitutive or regulated, as is the case, for example, when using pJOE2775 as expression plasmid with the addition of rhamnose. Expression of an alcohol dehydrogenase of the invention is followed by the purification thereof, for example by means of affinity chromatography or centrifugation. It is possible to use the thus purified enzymes but also the crude extracts or centrifugation supernatants or fractions directly for carrying out catalytic reactions.

Surprisingly, it was shown that the expressible proteins of the invention have enzymic alcohol dehydrogenase activity. The alcohol dehydrogenases of the invention reduce in particular cyclic, aromatic, aliphatic ketones and keto acids to the corresponding cyclic, aromatic, aliphatic alcohols and hydroxycarboxylic acids, respectively.

The claimed alcohol dehydrogenases are furthermore distinguished by their excellent stereoselectivity. Thus, for example, acetophenone is converted virtually exclusively, with 95% yield, to (R)-α-phenylethanol (>99% ee, determined by GC analysis) (in this context, see also Table 1). Thus, the present invention further relates to the use of alcohol dehydrogenases of the invention for catalytic preparation of alcohols or ketones, preferably of aliphatic, aliphatic-cyclic or arylaliphatic alcohols or ketones.

Particularly preferred substrates for the alcohol dehydrogenases of the invention are cyclic (C₃-C₁₀)-alkanones, (C₂-C₄₀)-keto acids and acetophenone derivatives, where the aromatic ring may contain substituents from the group consisting of H, (C₁-C₁₂)-alkyl, (C₁-C₁₂)-alkylO, F, Cl, Br, I, NR₂, COOH, preferably in 2, 3 and/or 4 position, where the substituents R are independently of one another H, (C₁-C₁₀)-alkyl or (C₃-C₁₀)-aryl.

The present invention further relates to a process for preparing alcohols with enzymic conversion of a ketone using an alcohol dehydrogenase of the invention.

The reduction is conducted preferably at between −10 and 45° C., particularly preferably between 5 and 25° C. (in this context, see FIG. 3). A particular surprise here is that ADHF1 achieves the highest yield with the substrate acetophenone at temperatures of from 10 to 25° C., although P. fluorescens is a mesophilic organism. The selectivities are very high across the entire temperature range and the highest selectivity with respect to the substrate acetophenone is reached at from 5° C. to 12° C. and from 37° C. to 42° C. The preferred pH for the enzymically catalyzed reduction of cyclic or aromatic ketones using the alcohol dehydrogenases of the invention is between pH 5.5 and pH 10, particularly preferably between pH 7 and pH 9 (in this context, see FIG. 4).

It is advantageous for carrying out the reduction of ketones to the corresponding alcohols using the alcohol dehydrogenases of the invention to add to the reaction mixture coenzymes supporting the reduction process, such as, for example, NADPH or preferably NADH. The addition of, for example, an NADH-recycling dehydrogenase may convert the NAD⁺ liberated during the conversion back into NADH.

Solvents which may be used may be both water and organic solvents or mixtures thereof. Examples of preferred solvents are, in addition to water, alcohols such as, for example, ethanol, or acetone. In order to regenerate NADH or NADPH, it is advantageous to add to the reaction mixture isopropanol which is oxidized to acetone in the process. Preference is given to a starting isopropanol content of from 2 to 35% (v/v), particularly preferably from 10 to 25%.

### BRIEF DESCRIPTION OF THE FIGURES

FIG. 1 depicts diagrammatically a physical map of a part region of the Pseudomonas fluorescens (DSM 50106) genome, comprising the coding region of an alcohol dehydrogenase of the invention (ORF3).

FIG. 2a shows an SDS-PAGE analysis (lanes 1 to 3) and an Ni-NTA-AP conjugation analysis (lanes 4 to 6) for detecting the successful expression of a P. fluorescens (DSM 50106) alcohol dehydrogenase of the invention. FIG. 2b shows an SDS-PAGE expression analysis for production of recombinant ADHF1 (296 aa, 31.997 kDa), lane M shows a marker for low molecular weights, Sigma, lane 1 shows a fractionated cell culture fraction prior to inducing expression, lane 2 shows such a fraction one hour after inducing expression and lane 3 shows a corresponding fraction after culturing has finished, lane 4 shows a concentrated supernatant (centrifugation), lane 5 shows a supernatant washed once, lane 6 shows a supernatant washed twice, lanes 7 and 8 show the cell pellet fraction.

FIG. 3 depicts the temperature effect on ADHF1 activity on the basis of selected substrates with respect to the yield a) for acetophenone (▴) and cyclohexanone (▪) and b) with respect to the yield (▴) and selectivity (▪) for acetophenone.

FIG. 4 depicts the pH effect on the enzymic activity of ADHF1 on the basis of the substrate acetophenone.

FIG. 5 depicts the solvent effect on ADHF1 activity on the basis of reduction of the substrate acetophenone to (R)-α-phenylethanol (FIG. 5a: yield: hatched bar, enantiomeric excess: light bar; FIG. 5b: yield (▴) and selectivity (▪)) as a function of isopropanol concentration.

Cloning and expression of an alcohol dehydrogenase from Pseudomonas fluorescens (DSM 50106).

General notes:

The host for transformation with DNA plasmids was the E. coli JM109 strain (Yanish-Perron et al. in Gene 33, 103-119 (1985)), and the hosts used for transformation with λRES phages were E. coli HB101 F′lac[Tn1739tnpR] strains (Altenbuchner, J. in Gene 123, 63-68 (1993)). The strains are cultured in LB liquid medium or on LB agar plates at 37° C. 100 μg/ml ampicillin or 50 μg/ml kanamycin are added to the media to select for plasmid-containing host cells. The vector used for cloning DNA sequences is plasmid pIC20H (Marsh et al. in Gene 32, 481-485 (1984)), and the vector used for rhamnose-inducible expression of ADHF1 in E. coli JM109 is plasmid pJOE2775, which contains the rhaBAD promoter (Stumpp et al. BIOspectrum 6, 33-36 (2000)).

Finding, sequencing and cloning of the adhF1 gene:

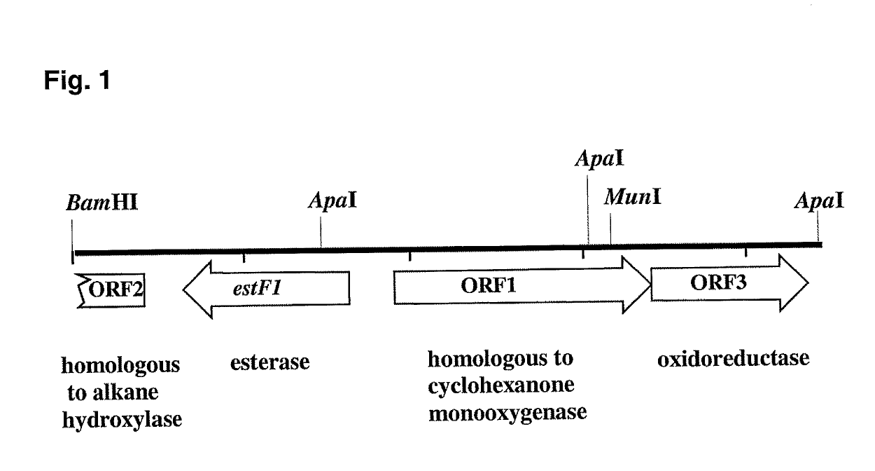

The construction of a genomic library of Pseudomonas fluorescens (DSM 50106) in λRESIII phages and the isolation of pJOE2967 plasmids showing esterase activity in E. coli are described in Khalameyzer et al.; Appl. Environm. Microbiol. 65, 477-482 (1999). In order to complement an open reading frame DNA sequence (ORF1 with homology to cyclohexanone monooxygenases; FIG. 1), an ApaI fragment overlapping over 150 bp was cloned, starting from a 3.2 kB MunI/BamHI fragment of vector pJOE2967 which displayed an esterase activity (estF1). Surprisingly, another open reading frame (OFR3; FIG. 1) was found on the ApaI restriction fragment, which, as can be shown, codes for a novel alcohol dehydrogenase. FIG. 1 depicts diagrammatically a 4360 bp physical map containing Pseudomonas fluorescens (DSM 50106) ORF3.

The P. fluorescens (DSM 50106) genomic region depicted in FIG. 1 was sequenced according to Sanger, pursuing two strategies. Firstly, NaeI and MscI restriction fragments were subcloned into plC20H. Furthermore, pFIS5 plasmids and deletion derivatives thereof were sequenced using Cy5-labeled M13 universal and reverse (UP and RP) primers and an ALFexpress AutoRead sequencing kit (Amersham Pharmacia Biotech). Primer walking was carried out using oligonucleotides from MWG Biotech, Ebersberg and Cy5-dATP labeled Nucleotids with the aid of the ALFexpress AutoRead sequencing kit (Amersham Pharmacia Biotech). The reaction products are fractionated in a 5.5% Hydrolink Long Ranger gel matrix in an ALFexpress DNA sequencer at 55° C. and 800V in 0.5×TBE buffer for 12 h. The nucleotide sequence was determined using the GCG program (Devereux et al., Nucleic Acid Res. 12, 387-395, (1984), version 8.01). A nucleic acid sequence depicted in Seq. Id. No. 2 was obtained for the adhF1 gene. DNA sequences which are 60% homologous to a putative oxidoreductase from Pseudomonas aeruginosa (Stover et al., Nature 406, 959-964, (2000)); GenBank accession number H83452 and 29% homologous to a C α-dehydrogenase from Pseudomonas paucimobilis (Masai et al., Biosci. Biotechnol. Biochem. 57, 1655-1659 (1993)) were found via a database search.

Standard methods described in Sambrook, et al. in Molecular cloning: a laboratory manual, NY: Cold Spring Harbor (1989), were used for restriction analysis and the cloning experiments. The plasmid DNA is isolated in analogy to Kieser, T. in Plasmid 12, 19-36 (1984). The restriction enzymes and DNA-modifying enzymes used therefor were from Boehringer Mannheim. E. coli are transformed according to Chung et al. in Proc. Natl. Acad. Sci. USA 86, 2172-2175 (1989).

In order to concentrate the ORF3-encoded DNA sequence (denoted adhF1 gene hereinbelow), a PCR amplification is carried out, starting from a plasmid containing the adhF1 gene and using the following PCR primers:

(SEQ ID NO. 3)

5′-AAA ACA TAT GAA GTC ATT CAA CGG CC-3′

(SEQ ID NO. 4)
5′-AAA AGG ATC CGA GAC GGG GCT CGT CGT T-3′

PCR amplification is carried out in a suspension of 1 ng of plasmid DNA, 30 pmol of primer, 0.2 mM dNTP mix, 10% DMSO, 2.5 units of Pwo polymerase in 1× reaction buffer (100 μl). For this purpose, the DNA is first heated at 100° C. for 2 minutes and then amplified in a minicycler (Biozym Diagnostics GmbH) in 30 cycles using a temperature program comprising denaturing at 94° C. for one minute, annealing the primers at a temperature which is 5° C. below the melting temperature of the primer for 1.5 minutes and polymerization at 72° C. for 1.5 minutes.

Into the amplification fragment obtained in this way, an Ndel restriction cleavage site was introduced directly in front of the ATG start codon and a BamHI restriction cleavage site was introduced directly in front of the stop codon. After Ndel and BamHI digestion of the thus modified amplification sequences, the DNA fragments obtained are cloned into an L-rhamnose-inducible expression vector pJOE3075 (Stumpp et al., BIOspectrum 6, 33-36 (2000)) which has likewise been subjected to an Ndel and BamHI digest. In this connection, the adhF1 gene region encoding the C-terminal end is fused to a vector tag encoding 6 histidines. Subsequently, an E. coli JM1 09 strain was transformed with the resulting plasmid pJOE4016 and cultured in 500 ml LB medium containing 100 μg/ml ampicillin at 37° C. until reaching the early exponential phase at OD₆₀₀=0.5 to 0.6.

Expression of ADHF1:

ADHF1 expression is induced by adding 0.2% (final concentration) rhamnose to the medium, and the cell culture is cultured at approx. 37° C. for 5 h. The cells obtained are centrifuged (Heraeus Labfuge 400R, 4000×g, 10 min, 4° C.) and washed twice with sodium phosphate buffer (50 mM, pH 7.5, 4° C.). The cells purified in this way are disrupted on ice with a 12-minute ultrasound treatment (50% pulse at 50% energy, Bandelin HD 2070, MS73, Berlin, Germany). The cell components are removed by centrifugation and the supernatant is either utilized directly for carrying out reduction reactions or lyophilized and stored at 4° C. The protein content is determined with the aid of the bicinchoninic acid kit (Pierce, Rockford, Ill., USA) with bovine serum albumin as protein standard. 450 μg of protein/mg of lyophilized extract were detected in the supernatant.

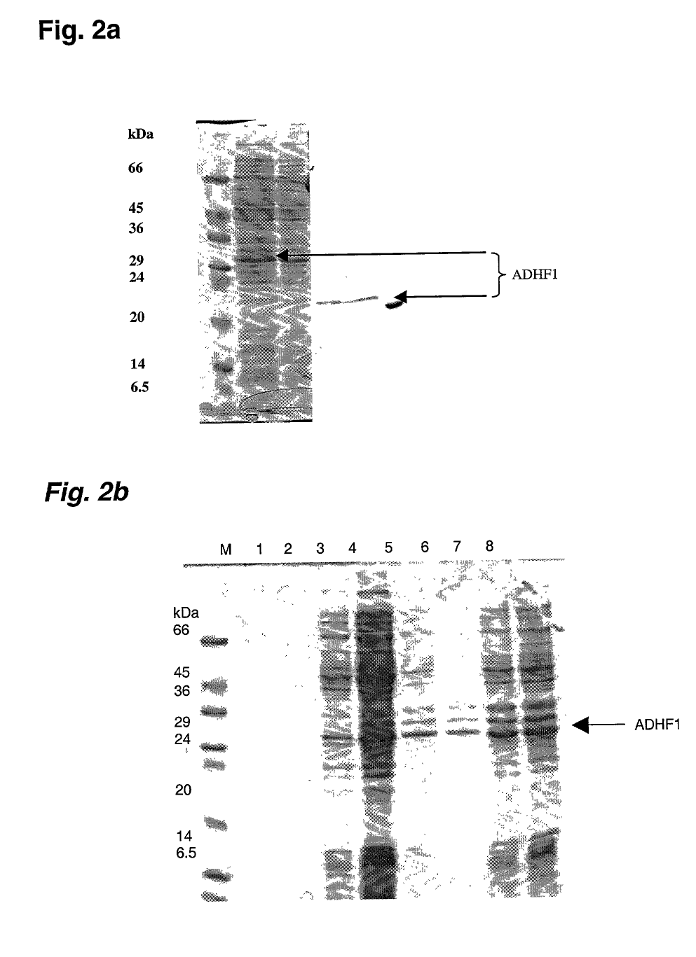

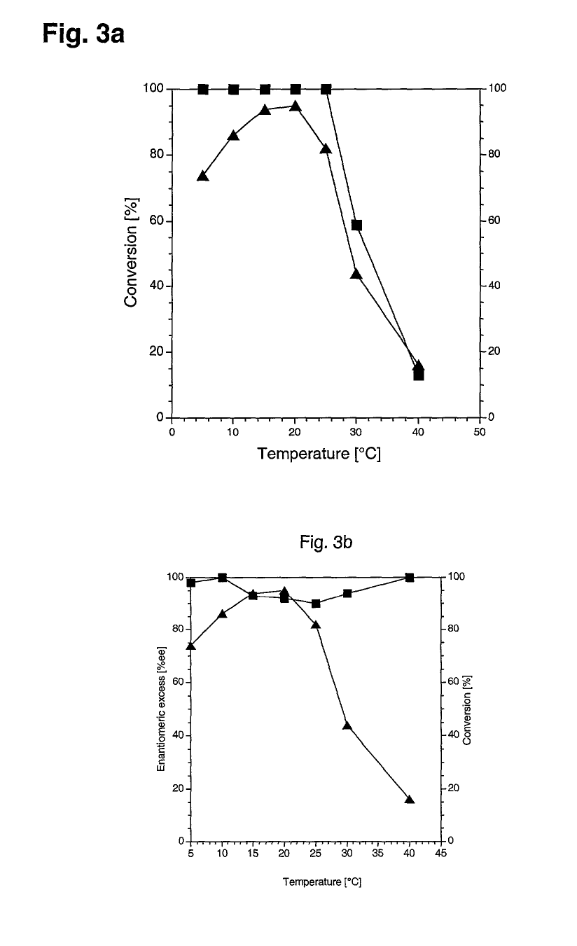

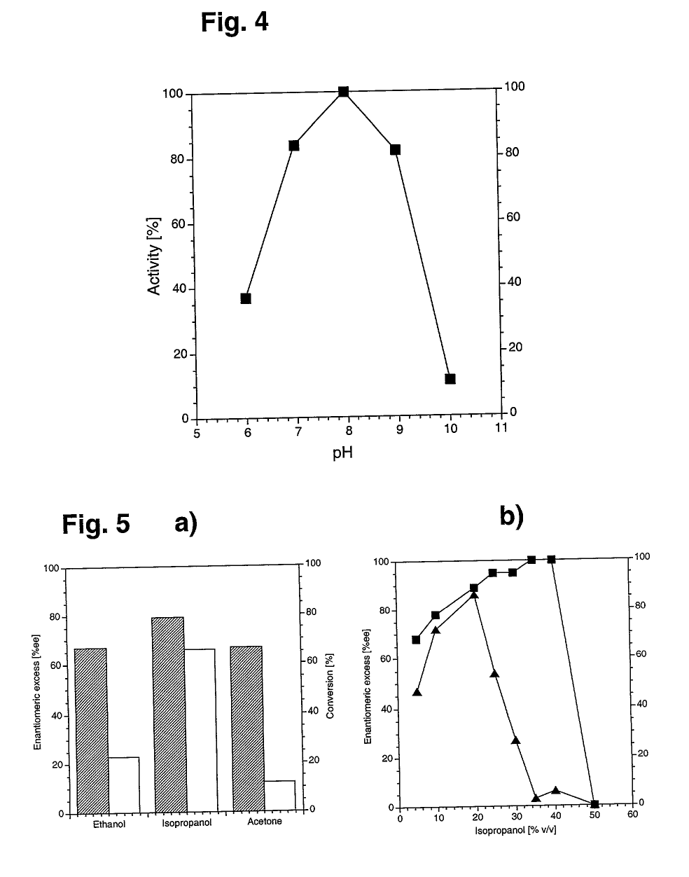

ADHF1 expression was checked by means of SDS-PAGE analysis (FIG. 2a, lanes 1 to 3) and by means of an Ni-NTA-AP conjugation assay (FIG. 2a, lanes 4 to 6). In detail, FIG. 2a depicts in: lane 1: standard (Sigma), lanes 2 and 4: 10 μg of ADHF1 crude extract, lanes 3 and 5: 5 μg of ADHF1 crude extract, lane 6: Sigma marker (carboanhydrase, 29 kDa, which reacts with Ni-NTA conjugates). FIG. 2b shows the result of another SDS-PAGE expression analysis of the production of recombinant ADHF1 (296 aa, 31.997 kDa); lane M shows a marker for low molecular weights, Sigma, the lanes 1, 2 and 3 show in each case a fractionated cell culture fraction prior to inducing expression, 1 hour after induction and after finishing culturing, the lanes 4, 5 and 6 show a concentrated supernatant, a supernatant washed once and a supernatant washed twice (centrifugation), the lanes 7 and 8 show in each case cell pellet fractions. The proteins in the SDS-PAGE gel were stained directly with Coomassie brilliant blue or gel-electrophoretically blotted on a nitrocellulose membrane at 1 mA/cm² for 1 h using a semi-dry blotting system (Panther, semi-dry electroblotter, Model HEP-1, Peqlab, Erlangen). The histidine-labeled proteins are detected on nitrocellulose according to the manufacturer's instructions (QIApress detection system, Ni-NTA alkaline phosphate conjugates, Qiagen, Hilden). Carboanhydrase (29 kDa) is used as Ni-NTA conjugate-binding mass standard (lane 3, FIG. 2a).

Use of the expressed alcohol dehydrogenase for the reduction of ketones:

The activity of lyophilized alcohol dehydrogenase is determined using acetophenone as model substrate. The standardized reaction mixture (250 μl) contains 6.4 μmol of substrate dissolved in isopropanol, 1.25 mg of enzyme (crude extract) in 0.1 M Tris buffer (pH 8.0) and 20% (v/v) isopropanol as substrate for NADH-recycling dehydrogenase. The reactions are carried out at room temperature, unless explicitly stated otherwise. All values were determined three times. After the reaction has finished, the reaction mixture is extracted with twice the amount of chloroform and the organic phase is then dried over sodium sulfate. The reaction products were analyzed isothermally at 120° C. by GC measurements (Shimadzu GC 14A, Tokyo, flame ionization detector, integrator C5RA) using a chiral column (heptakis (2,6-O-methyl-3-O-pentyl)-β-cyclodextrin, 25m×0.25mm ID, Macherey & Nagel, Düren). The retention times are 1.55 min for acetophenone, 2.77 min for (R)-α-phenylethanol and 2.99 min for (S)-α-phenylethanol. The absolute configuration was determined by using commercial standards. Further GC analyses were carried out at a temperature rising from 90° C. to 120° C. at a rate of 5° C./min. Here the retention times were 0.95 min for cyclopentanone, 1.37 min for cyclopentanol, 2.85 min for cycloheptanone, 4.29 min for cycloheptanol, 1.42 min for cyclohexanone and 2.12 min for cyclohexanol.

The results of the conversions are listed in Table 1.

TABLE 1

| | Conversion^{a} | Time |
|---|---|---|
| Substrate | [%] | [h] |
| Cyclopentanone | 53 | 20 |
| Cyclohexanone | 100 | 20 |
| Cycloheptanone | 51 | 20 |
| Acetophenone | 95^{b} | 21 |

^{a}Determined by GC analysis;
^{b}>99% ee (R)-α-phenylethanol

As a control, crude extracts of E. coli strains containing no recombinant ADH gene were likewise tested. These extracts show no corresponding enzymic activity.

Table 2 shows the results of further reaction mixtures converted according to the protocol above (in this context, see also diagram 1):

TABLE 2

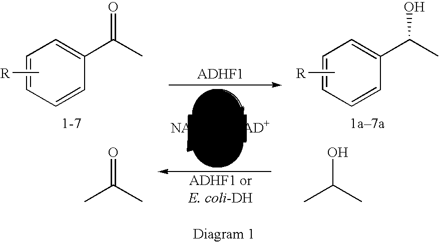

| | Conversion^{b} | Enantiomeric | Time |
|---|---|---|---|
| Substrate/R^{a} | [%] | excess [%ee]^{b} | [h] |
| 1/H | 95 | 92 | 21 |
| 2/4-Me | 82 | 42 | 19 |
| 3/2-MeO | 31 | >99 | 21 |
| 4/3-MeO | 89 | 92 | 19 |
| 5/4-MeO | 38 | 45 | 20 |
| 6/4-F | 91 | 91 | 21 |
| 7/4-Cl | 29 | 79 | 19 |
| 8^{c} | 83 | >99 | 19 |

^{a}R for 1-7 as in diagram 1;
^{b}determined by GC analysis;
^{c}methyl 3-oxobutyrate

For a temperature program starting from 120° C. (2 min) and subsequent heating by 10° C./min up to 150° C., the following retention times are obtained: 2-methoxy-acetophenone 3, 3.68 min, 2-methoxy-α-(R)-phenylethanol 4.90 min, 3-methoxyacetophenone 4, 3.98 min, (R)-3-methoxy-α-phenylethanol 4a, 5.61 min, (S)-4a, 5.86 min; 4-fluoroacetophenone 6, 1.58 min, (R)-fluoro-c-phenylethanol 6a, 3.03 min, (S)-6a, 3.28 min. For a temperature program starting from 90° C. (1 min) and subsequent heating by 5° C./min up to 120° C., the following retention times are obtained: 4-chloro-acetophenone 7, 3.62 min, (R)-4-chloro-α-phenylethanol 7a, 5.52 min, (S)-7a, 5.90 min; 4-methylacetophenone 2, 2.63 min, (R)-4-methyl-α-phenylethanol 2a, 3.40 min, (S)-2a, 3.76 min; 4-methoxyacetophenone 5, 4.94 min, (R)-4-methoxy-α-phenylethanol 5a, 5.52 min, (S)-5a, 5.74 min. An isothermal GC analysis at 70° C. results in the following retention times: methyl 3-oxobutyrate 8, 2.20 min, methyl (R)-3-hydroxybutyrate 8a, 3.84 min, (S)-8a, 3.99 min.

The absolute configuration of la and 8a was verified via the commercial (R) alcohols and it is assumed that elution of the acetophenone analogs is in the same order (R before S) as that of α-phenylethanol.

Determination of the solvent effect on ADHF1 activity:

For this purpose, isopropanol, acetone or ethanol were added to the reaction mixture at a final concentration of 10% (v/v). Additionally, the isopropanol proportion of the acetone reaction mixture was varied. The substrate used was acetophenone. The measurements were carried out at 20° C. and the other reaction conditions were taken from the protocol above. The results are graphically depicted in FIG. 5.

Patent claims:

1. An alcohol dehydrogenase (ADHF1) from Pseudomonas fluorescens (DSM 50106), having the amino acid sequence Seq. Id. No. 1 or an allelic or functional variant thereof or a functional part sequence thereof.

2. The alcohol dehydrogenase as claimed in claim 1, comprising an amino acid sequence which is more than 60% homologous to Seq. Id. No. 1.

3. The alcohol dehydrogenase as claimed in claim 1, comprising an amino acid sequence which is more than 80% homologous to Seq. Id. No. 1.

4. The alcohol dehydrogenase as claimed in claim 1, comprising a part sequence Seq. Id. No. 1 or an allelic or functional variant thereof composed of at least 50 amino acids.

5. The alcohol dehydrogenase as claimed in claim 1, comprising a part sequence of Seq. Id. No. 1 or an allelic or functional variant thereof having deletions of up to 100 amino acids.

6. An alcohol dehydrogenase gene from Pseudomonas fluorescens (DSM 50106), having a coding nucleic acid sequence Seq. Id. No. 2 and allelic or functional variants thereof which are more than 50% homologous or part sequences thereof.

7. The alcohol dehydrogenase gene as claimed in claim 6, comprising a nucleic acid sequence which is more than 75% homologous to Seq. Id. No. 2.

8. The alcohol dehydrogenase gene as claimed in claim 6 or 7, comprising a part sequence of Seq. Id. No. 2 with at least 150 nucleotides.

9. The alcohol dehydrogenase gene as claimed in any of claims 6 to 8, comprising a nucleic acid sequence which is complementary to those nucleic acid sequences hybridizing with a coding nucleic acid Seq. Id. No. 2 or an allelic or functional variant or part sequences thereof under stringent conditions.

10. A vector, comprising a nucleic acid sequence as claimed in any of claims 6 to 9.

11. An expression system, comprising a host cell and at least one vector as claimed in claim 10.

12. An expression system, wherein the host organism is a yeast or a prokaryote.

13. The use of alcohol dehydrogenases as claimed in any of claims 1 to 5 for enzymically reducing ketones to the corresponding alcohols.

14. A process for reductive preparation of alcohols from ketones, wherein the ketone is converted using an alcohol dehydrogenase as claimed in claims 1 to 5.

15. The process as claimed in claim 14, wherein the conversion is carried out at between −10 and 45° C.

16. The process as claimed in either of claims 14 and 15, wherein the conversion is carried out at between pH 5.5 and 10.

17. The process as claimed in any of claims 14 to 16, wherein further auxiliary substances are added to the reaction mixture.

18. The process as claimed in claim 17, wherein the auxiliary substances added are NADH and/or NADPH.

19. The process as claimed in claim 18, wherein an NADH- and/or NADPH-recycling dehydrogenase are added to the reaction mixture.

20. The process as claimed in claim 19, wherein between 2 and 35% (v/v) isopropanol are added to the reaction mixture.

21. The process as claimed in any of claims 14 to 20, wherein the substrate used is aliphatic, cyclic, aromatic, aromatic-aliphatic ketones or keto acids.

22. The process as claimed in any of claims 14 to 21, wherein the substrates used are cyclic (C₃-C₁₀)-alkanones, (C₂-C₄₀)-keto acids and acetophenone derivatives, where the aromatic ring has substituents from the group consisting of H, (C₁-C₁₂)-alkyl, (C₁-C₁₂)-alkylO, F, Cl, Br, I, COOH, NR₂, where the residues R may be independently of one another H, (C₁-C₁₀)-alkyl or (C₃-C₁₀)-aryl.


SEQUENCE LISTING (4 sequences)

SEQ ID NO: 1 - protein, 296 aa, Pseudomonas fluorescens
MKSFNGRVAAITGAASGMGRALALALAREGCHLALADKNAQGLEQTLALIKTSTLSPVMVTTQVLDVADRQAMEAWAARCVAEHGQVNLVFNNAGVALSSTVEGVDYADLEWIVGINFWGVVHGTKAFLPHLKASGDGHVINTSSVFGLFAQPGMSGYNATKFAVRGFTEALRQELDLQRCGVSATCVHPGGIRTDICRSSRIDANMTGFLIHSEQQARADFEKLFITDADQAAKVILQGVRKNKRRVLIGRDAYFLDLLARCLPAAYQALVVLASKRMAPKQRRPVFETNDEPRL

SEQ ID NO: 2 - DNA, 891 bp, Pseudomonas fluorescens
atgaagtcattcaacggccgcgtggcggcgattaccggcgcggcatccggcatgggtcgcgcaatggccctggcactcgcgcgcgaaggttgccacctggcactggcggacaaaaacgcccaaggcctggagcagaccctggcactgatcaagacctcgaccctgtcgccggtgatggtcaccacccaggtgctggatgtggccgaccgccaggccatggaggcttgggcggcgcgctgcgtggccgagcatggccaggtcaacctggtgttcaacaacgccggcgtggccctgtcgagtacggtcgaaggcgtggactacgccgacctggagtggatcgtcggcatcaacttctggggcgtggtccacggcaccaaggcgttcctgccgcacctcaaggccagcggcgacggccatgtgatcaacacgtccagcgtgttcggcctgtttgcccagcccggcatgagcggttacaacgcgaccaaattcgccgtgcgcggctttaccgaagccctgcgccaggagctggacctgcaacgctgcggcgtctcggccacctgcgtgcaccccggcggcatccgcaccgatatctgtcgcagcagccgcatcgacgcgaacatgaccggcttcctgatccacagcgaacagcaggcccgcgccgacttcgaaaaactcttcatcaccgatgccgaccaggccgccaaggtgatcctgcaaggcgtccgcaaaaacaagcgtcgcgtgctgataggccgcgacgcgtatttcctcgacctgctcgcccgttgcctgccggcggcctatcaagcgctggtggtgctggccagcaagcgcatggcccccaagcaacgcaggccagtgtttgaaaccaacgacgagccccgtctctga

SEQ ID NO: 3 - DNA, 25 bp, Artificial Sequence
  synthetic DNA
aaacatatgaagtcattcaacggcc

SEQ ID NO: 4 - DNA, 28 bp, Artificial Sequence
  synthetic DNA
aaaaggatccgagacggggctcgtcgtt